(19) United States
(12) Patent Application Publication
(10) Pub. No.: US 2024/0399038 A1
(43) Pub. Date: Dec. 5, 2024

(54) DIALYSIS FLUID GENERATION SYSTEM USING RECIRCULATION

(71) Applicants: Baxter International Inc., Deerfield, IL (US); Baxter Healthcare SA, Glattpark (Opfikon) (CH)

(72) Inventors: James White, Grayslake, IL (US); Jonas Fors, Malmö (SE); Karl Henrik Forsland, Genarp (SE); Unberto Garcia, Bellwood, IL (US); Steven Hanson, Antioch, IL (US); Scott Kopacek, Cary, IL (US)

(21) Appl. No.: 18/694,800

(22) PCT Filed: Sep. 6, 2022
(86) PCT No.: PCT/EP2022/074725

§ 371 Date: Mar. 22, 2024

Related U.S. Application Data
(60) Provisional application No. 63/247,968, filed on Sep. 24, 2021.

(51) Int. Cl. A61M 1/16

(52) U.S. Cl. CPC A61M 1/1694

(57) ABSTRACT

A dialysis fluid production system includes a water purification unit, at least one concentrate, a dialysis machine, a product container, and a recirculation container. The dialysis machine is configured to receive purified water from the water purification unit and to mix the purified water with the at least one concentrate to form dialysis fluid. The product container is positioned and arranged to receive the dialysis fluid from the dialysis machine, and the recirculation container is configured to receive an end of the drain line and an end of the dialysis fluid return line. Further, the recirculation container enables dialysis fluid to be recirculated from the drain line, through the dialysis fluid return line, back to the dialysis machine and is configured to be positioned adjacent to a facility drain such that overflow dialysis fluid flows from the recirculation container into the facility drain.

### BACKGROUND

The present disclosure relates generally to medical fluid generation and in particular to dialysis fluid generation.

Due to various causes, a person's renal system can fail. Renal failure produces several physiological derangements. It is no longer possible to balance water and minerals or to excrete daily metabolic load. Toxic end products of metabolism, such as, urea, creatinine, uric acid and others, may accumulate in a patient's blood and tissue.

Reduced kidney function and, above all, kidney failure is treated with dialysis. Dialysis removes waste, toxins and excess water from the body that normal functioning kidneys would otherwise remove. Dialysis treatment for replacement of kidney functions is critical to many people because the treatment is lifesaving.

One type of kidney failure therapy is Hemodialysis (“HD”), which in general uses diffusion to remove waste products from a patient's blood. A diffusive gradient occurs across the semi-permeable dialyzer between the blood and an electrolyte solution called dialysate or dialysis fluid to cause diffusion.

Hemofiltration (“HF”) is an alternative renal replacement therapy that relies on a convective transport of toxins from the patient's blood. HF is accomplished by adding substitution or replacement fluid to the extracorporeal circuit during treatment. The substitution fluid and the fluid accumulated by the patient in between treatments is ultrafiltered over the course of the HF treatment, providing a convective transport mechanism that is particularly beneficial in removing middle and large molecules.

Hemodiafiltration (“HDF”) is a treatment modality that combines convective and diffusive clearances. HDF uses dialysis fluid flowing through a dialyzer, similar to standard hemodialysis, to provide diffusive clearance. In addition, substitution solution is provided directly to the extracorporeal circuit, providing convective clearance.

The corresponding continuous treatment modalities of HD, HF and HDF, namely, continuous venous vivo (“CVV”) CVVHD, CVVH and CVVHDF are each modalities of continuous renal replacement therapy (“CRRT”), which may be used to treat acute kidney injury (“AKI”). AKI is more common than most people know and is under-recognized in hospital patients, especially in certain countries. AKI may be brought on by disease, such as COVID-19, which is caused by SARS-COV-2. This may occur with patients who had no underlying kidney problems before being infected with SARS-COV-2. Certain reports have stated that up to 30% of patients hospitalized with COVID-19 in China and New York have developed moderate to severe kidney injury. Signs of kidney problems in patients with COVID-19 include high levels of protein in the urine and abnormal blood work.

During the early stages of the COVID-19 pandemic in the United States, a spike in the use of CRRT solutions occurred as many COVID-19 patients in the ICU experienced acute renal failure. When plant produced solutions have come into short supply during the COVID-19 pandemic, doctors have resorted to several alternate methods to sustain the patient's renal clearance and keep them alive. In some cases, hospitals resorted to off label use of HD machines in the ICU or in the hospital's HD clinic to produce bagged dialysis fluid for CRRT treatment.

To convert the HD machines into dialysis fluid producing machines, the drain line from the dialysis machine and the dialysis fluid return line that normally runs from the dialyzer back to the dialysis machine are both extended from the dialysis machine to a recirculation container. Dialysis fluid is pulled from the recirculation container to the dialysis machine through the dialysis fluid return line at the same rate that the dialysis fluid is pushed to the recirculation container from the dialysis machine through the drain line. In this manner, the level of dialysis fluid within the recirculation container during the production of bagged dialysis fluid remains constant. In this way, the safety mechanisms of the dialysis machine intended for balanced fluid flow during an HD treatment are mechanically worked-around, preventing alarms and allowing the machine-produced dialysis solution to flow to a product bag

When a bag becomes full and needs to be removed, capped and replaced, the production of dialysis fluid is stopped temporarily and the dialysis machine is placed in a bypass mode in which dialysis fluid is no longer pulled from the recirculation container and along the dialysis fluid return line into the dialysis machine. The flow of dialysis fluid from the machine to the recirculation container through the drain line is maintained however, resulting in an increase in the level of dialysis fluid within the recirculation container.

In the known off-label production of dialysis fluid for CRRT, the recirculation containers are placed in a large, open facility drain within the hospital. Dialysis fluid is allowed to overflow from the recirculation containers into the common drain when dialysis fluid accumulates over multiple bypass modes.

Allowing dialysis fluid to overflow from the recirculation containers is not recommended for a temporary larger scale, e.g., mobile, preparation unit configured to produce dialysis fluid for CRRT. Drains large enough to accommodate multiple recirculation containers create contamination issues. Dialysis fluid is a high microbial growth media. The filling of containers with dialysis fluid requires aseptic precautions to be implemented and followed carefully. Spills, large open drains with dialysis fluid, and the like, present contamination issues for a dialysis fluid production environment.

Because dialysis fluid flows from the recirculation containers back to the HD machine, if the recirculation containers contain adherent biofilm, contamination from the film will eventually migrate to the HD machine. For this reason, the recirculation containers need to either be cleaned for reuse or discarded after use.

Requiring the system operators to periodically empty or change out the recirculation containers also presents problems. If the recirculation containers are larger in size they are less prone to toppling and spilling but are harder to maneuver and create storage issues. If the recirculation containers are smaller in size they are easier to manually maneuver but are more prone to toppling and spilling, creating contamination issues within the dialysis fluid production unit.

Additionally, if the recirculation containers are durable they need to be carefully cleaned on a periodic basis, adding stress and strain on the operators and slowing production. If the recirculation containers are instead disposable, disposable cost and storage may become an issue. Additionally, if the disposable containers need to be modified at all for use with the dialysis fluid production, such modification is cumbersome when many disposable recirculation containers are needed and consumed.

Each of the above scenarios is complicated by the handling of lines in addition to the dialysis fluid return line and the drain line that have to be routed to drain near the recirculation container. Such lines add to any existing complexity in the handling of the recirculation containers.

A need accordingly exists for a dialysis fluid preparation unit and associated system that addresses the above-described issues.

### SUMMARY

The present disclosure sets forth a dialysis fluid production systems that may, but do not have to be, used in an emergency or temporary situation in which a shortage of dialysis fluid exists, e.g., during a pandemic in which the disease results in a high incidence of acute kidney injury. The system is in one embodiment mobile, e.g., provided in a cargo unit or other self-contained, transportable modality, which may be loaded onto a flatbed truck or railroad car for transportation, or which may be constructed on wheels to be attached to a truck directly. As discussed below, the system may be implemented in many different locations/situations and is not limited to a cargo or other self-contained unit.

Inside the cargo unit, a plurality of dialysis fluid preparation units are provided. The dialysis fluid preparation units include dialysis machines that operate with water purification equipment. The cargo unit receives tap water, e.g., via an external hose, which may be run through a pretreatment subsystem and water purification units and delivered to the dialysis machines. The dialysis machines mix the purified water with concentrates, such as an acid concentrate and a bicarbonate concentrate to form a dialysis fluid.

In the emergency production system, each dialysis machine outputs through a line, such as a flexible tube, to a dialysis fluid connector of a filter, such as an ultrafilter (or perhaps a dialyzer). An “inside-out” type of filtration provided by an ultrafilter is preferred although an “outside-in” type of filtration provided by a dialyzer would work if its hollow fibers are not damaged by the resulting backpressure of the dialysis fluid. The ultrafilter (or dialyzer) membranes may act as a final stage of purification prior to the bagging of the dialysis fluid. The dialysis fluid exits the dialyzer through a line, such as a flexible tube, and flows through an adapter connected to a presterilized and empty product container or bag, where the dialysis fluid is stored for use.

Each of the structures above, including the water purification equipment (pretreatment subsystem and water purifier), dialysis machines, concentrates and disposable items may be provided within a cleanroom that may be positioned in any desirable area of the cargo unit. Within that cleanroom in one embodiment is a laminar HEPA air flow station under which the dialysis fluid is transferred from the dialysis machine via the adapter to the bag. The laminar HEPA air flow provides additional protection against pathogens entering the presterilized product container or bag during the connection step, during which time the flow path is exposed to the ambient environment.

In normal clinical operation (e.g., during a hemodialysis (“HD”) treatment), the dialysis machine delivers fresh dialysis fluid to a dialyzer and removes used dialysis fluid from the dialyzer. The used dialysis fluid is delivered to drain. It is common across multiple manufacturers of dialysis machines to require the used dialysis fluid pump to pump used dialysis fluid from the dialyzer to drain in order for the fresh dialysis fluid pump to pump fresh dialysis fluid to the dialyzer. Such balanced flow is provided to control an ultrafiltration rate, or the amount of fluid slowly removed from the patient during the HD treatment. So for the present dialysis fluid preparation application, it is not possible to only run the fresh dialysis fluid pump, the concentrate pumps and the purified water pump without running the used dialysis fluid pump. The used dialysis fluid pump needs to be operated as well and its flowrate may be approximately in balance with the fresh dialysis pump.

To enable the used dialysis fluid pump to be operated during operation of the present system, the system provides a recirculation container that receives both the distal end of the drain line and the end of the dialysis fluid return line that is normally connected to the dialyzer. The drain line in one embodiment terminates with a drain hook, which hooks over the side wall of the recirculation container. The dialysis fluid return line in one embodiment extends to a bottom of the recirculation container.

At the startup of a production shift, each dialysis machine is primed with dialysis fluid. The entire dialysis fluid circuit including the water lines, the concentrate lines, the fresh dialysis fluid lines, and the used dialysis fluid lines are primed. The flexible fresh dialysis fluid line to the filter and the filter are primed. Additionally, the flexible dialysis fluid return line and the drain line are primed, leaving a volume of dialysis fluid within the recirculation container. In an embodiment, the end of the dialysis fluid return line is submerged within the volume of dialysis fluid, while the drain hook at the end of the drain line hangs above the volume of dialysis fluid residing in the recirculation container.

During the production of dialysis fluid, as dialysis fluid is filling a presterilized bag, the used dialysis fluid pump pulls the priming dialysis fluid from the recirculation container into the dialysis machine via the dialysis fluid return line and pushes the dialysis fluid from the dialysis machine to the recirculation container via the drain line. In this manner, the dialysis fluid used for priming is circulated in a closed loop between recirculation container and the dialysis machine as a bag is being filled with freshly prepared dialysis fluid.

When the bag is full of dialysis fluid and needs to be removed and replaced with a new empty presterilized bag, the operator presses a bypass button on the dialysis machine. Pressing of the bypass button causes the valve state within the dialysis machine to switch. The bypass valve state blocks the return flow of dialysis fluid from the recirculation container into the dialysis machine along the dialysis fluid return line. The bypass valve state also causes newly prepared dialysis fluid to be diverted to drain instead of flowing to the ultrafilter. The bypass valve state accordingly creates a situation in which dialysis fluid flows into the recirculation container via the drain line but does not flow back to the dialysis machine via dialysis fluid return line. The level of dialysis fluid in the recirculation container increases accordingly.

The cargo unit is provided with one or more facility drain, which may for example be a three inch diameter drain that funnels down to a smaller diameter tube extending up from the floor of the cargo unit. In one primary embodiment, the recirculation container is a beaker, e.g., plastic or glass, which is provided as part of a recirculation assembly along with a recirculation fixture. The recirculation fixture does not touch fluid and is a durable component that is reused over multiple production shifts. The recirculation container does touch dialysis fluid as discussed and is a disposable component that is discarded after each shift. The recirculation containers may be packaged in sealed pouches and then sterilized for use in the cleanroom.

The recirculation fixture is plastic in one embodiment and may be molded or made using an additive manufacturing process. The recirculation fixture includes a drain mount sized to fit over and rest securely on the top the cargo unit's facility drain. The drain mount may be in the form of a flanged ring sized to fit around the largest outer diameter of the facility drain. A container holder extends from the drain mount. The container holder may have a cylindrical or ring shape sized to frictionally receive the recirculation container; and/or the recirculation container may be provided with a lip extending around the open end of the container, wherein the lip rests against the container holder so as to hold the recirculation container in a set position. The container holder extends at an angle relative to the drain mount, wherein the angle is selected so as to allow an amount of dialysis fluid to be held within the recirculation container that is sufficient to fully submerge the end of the dialysis fluid return line while filtered dialysis fluid is filling a bag and the used dialysis fluid pump is recirculating dialysis fluid from the recirculation container back to the dialysis machine. The angle is also selected so that during the bypass valve state when dialysis fluid accumulates in the recirculation container, the dialysis fluid overflows cleanly from a spout of the recirculation container into the cargo unit's facility drain without running down the outside of the recirculation container and creating drips within the cleanroom.

The angle between the container holder and the drain mount is dependent on the angle of the spout and the surface tension of the dialysis fluid. It has been found that holding the recirculation container such that its spout extends about 9° above horizontal provides sufficient dialysis fluid volume accumulation during dialysis fluid recirculation (to submerge the end of the dialysis fluid return line), while preventing dialysis fluid drops from forming and flowing along the outside of the recirculation container, e.g., in a non-overflow or static situation, which could create a contamination risk. In one specific example using a beaker manufactured by Globe Scientific, part #BPM1000P, an angle of 28.6° between container holder and the drain mount causes the container holder to hold the beaker such that the bottom edge of the spout is oriented roughly 9° degrees above horizontal. It should be appreciated that different recirculation containers with different spouts may require different angles between the container holder and the drain mount, however, it is believed that an angle between and including 20° to 40° should work with virtually all recirculation containers of the first primary embodiment.

The size of the recirculation containers may be on the order of one liter or less. The container holder is sized to hold the recirculation container firmly throughout a production shift but to allow the recirculation container to be removed easily at the end of the shift, e.g., by sliding out from the container holder. One or both of the container holder and the drain mount of the recirculation fixture may be provided with securement features for contacting and orienting the various lines running to the recirculation container and to the cargo unit's facility drain. In one example, the container holder includes securement features for contacting and orienting the dialysis fluid return line and the drain hook of the drain line, which each extend into the recirculation container. In one implementation, one of the container holder securement features holds the dialysis fluid return line so that an end of line may extend to the bottom of the recirculation container. The other of the container holder securement features holds the drain hook of the drain line such that an outlet of the drain line resides above the level of dialysis fluid within the recirculation container during recirculation.

The drain mount of the recirculation fixture may also include one or more securement feature. For example, the drain mount may include a first securement feature for contacting and orienting a priming line that extends from a top of the ultrafilter. A second securement feature may be provided to contact and orient a water purification unit drain line that extends from the water purification unit. The priming line and the water purification unit drain line both extend to the cargo unit's facility drain rather than the recirculation container, so their associated securement features are provided by the drain mount, which rests atop the cargo unit's facility drain.

A separate cargo unit facility drain may be provided for each dialysis fluid preparation unit (including dialysis machine and water purification unit). Or, a single facility drain may be provided for multiple dialysis fluid preparation units. In such a case, the recirculation fixture may include multiple container holders molded in place with drain mount. Here, the drain mount may include securement features for multiple priming lines and multiple water purification unit drain lines. The multiple container holders each hold a recirculation container for a dedicated dialysis machine and are provided with securement features for the dialysis fluid return line and the drain hook of the drain line as discussed.

It should be appreciated that the recirculation fixture may be configured to hold recirculation containers having a different shape than the beaker just described. The recirculation containers may instead be more closed and provide multiple openings for the dialysis machine drain line and dialysis fluid return line. Examples of such containers include beverage dispensers, squeeze and pour containers and tip and pour containers. Such containers may operate with dedicated and optimized support fixtures for orienting the containers in a desired manner relative to the facility drain.

In a second primary and alternative embodiment, the recirculation container instead includes a closed chamber, which may be a disposable part formed from a blow or injection molding process, for example, an existing HD expansion chamber accessory. When oriented for operation in a generally vertically extending configuration, the closed chamber includes an upper closed chamber line extending vertically upward from a top of a closed chamber body and two lower closed chamber lines extending vertically downward from a bottom of the closed chamber body.

With the closed chamber body fixed in the generally vertical orientation to a side of the cargo unit's facility drain, the upper closed chamber line may be bent 180° and extended into the facility drain. One of the two lower closed chamber lines is connected and scaled fluidically to the dialysis machine drain line, e.g., via a hose clamp. The other of the two closed chamber lines is connected and sealed fluidically to the dialysis fluid return line, e.g., via an intermediate line segment that includes the correct counterpart connectors for the closed chamber line and the dialysis fluid return line.

The closed chamber body has a small volume, which remains at least partially full during dialysis fluid recirculation between the closed chamber body and the dialysis machine. When the dialysis machine is switched to the bypass valve state, dialysis fluid overflows from the closed chamber body, through the upper closed chamber line, to the cargo unit's facility drain. The closed chamber may be provided in a presterilized package and be discarded at the end of a production shift. Due to the small volume and enclosed nature of the closed chamber body, however, it may be possible to instead disinfect the closed chamber along with the dialysis machine at the end of treatment. In this manner, the same closed chamber may be used as the dialysis fluid recirculation container over multiple production shifts.

In a third primary and alternative embodiment, the recirculation container is configured to also allow it to be secured to the cargo unit's facility drain. So like with the second primary embodiment (closed chamber), the durable or reusable recirculation fixture of the first primary embodiment is removed. In the third primary embodiment, the recirculation container includes an inner wall and an outer wall. The inner wall abuts up against the outer diameter of the facility drain. The outer wall is set back from the inner wall a distance sufficient to create, along with the depth of the container, a desired volume for a dialysis fluid holding space. The inner and outer walls may be curved to follow the outer diameter of facility drain. The inner and outer walls may be sized to allow multiple recirculation containers, for multiple dialysis machines and dialysis fluid preparation units, to be fitted to the same facility drain at once.

The outer wall in one embodiment extends up from dialysis fluid holding space a distance sufficient to hold the drain hook of the dialysis fluid drain line so that dialysis fluid exits the drain hook above the dialysis fluid level. The outer wall may be provided with a notch for holding the drain hook so that it cannot slide along the top of the outer wall. The inner wall forms a weir or dam structure, which (i) provides a mounting hook that enables the recirculation container to be removably fixed to the facility drain and (ii) is the lowest wall of the container such that dialysis fluid overflows out of the recirculation container, over the inner wall, and into the facility drain. The weir is in one embodiment curved along with the inner wall to follow the outer diameter of facility drain.

A return tube extends from a bottom of the recirculation container of the third primary embodiment and is fitted at its end with a connector, e.g., male Hansen connector, which is configured to connect to the connector, e.g., female Hansen connector, located at the end of the dialysis fluid return line of the HD machine. Here, the connector at the end of the dialysis fluid return line does not have to be submerged in the recirculation container, and it is ensured that the intake to the dialysis fluid return line is at the lowest point of the recirculation container. During recirculation, dialysis flows between the recirculation container and the dialysis machine and a constant level of dialysis fluid is maintained within the dialysis fluid holding space of the recirculation container. When the dialysis machine is in the bypass state, the level of dialysis fluid rises and eventually overflows over the inner wall and weir into the facility drain.

Other than the notch in the outer wall, the recirculation container of the third primary embodiment does not need securement features, such as for the dialysis fluid return line since it is attached to the return tube of the recirculation container. The recirculation container may provide one or more securement feature for the priming line and the water purification unit drain line, which would be held by the fixtures and extend past the recirculation container and into the cargo unit's facility drain. The recirculation container of the third primary embodiment is in one embodiment packaged in a pouch, presterilized, and discarded after a product shift.

In addition to the weir recirculation container just described, other recirculation containers where the connection to the facility drain also serves as the location where overflow dialysis fluid flows into the facility drain may include vacuum flasks, wash bottles and plastic piping.

In a fourth primary and alternative embodiment, the recirculation container is instead a larger, durable recirculation container that is reused and periodically cleaned, e.g., after each production shift. In one embodiment, the dialysis machine drain line extends to an upper container connector, while the dialysis fluid return line extends to a lower container connector, such that dialysis fluid enters the container above a level of dialysis fluid and so that there is plenty of head of dialysis fluid to feed the dialysis fluid return line. In an alternative embodiment, say if the upper container connector is a matching connector for that of the dialysis fluid return line, then the lines may be reversed with the dialysis fluid return line connecting to the upper container connector and the dialysis machine drain line connecting to the lower container connector. Here, a straw extending from the upper connector to the bottom of the container may be provided inside of the container so that the inlet to the dialysis fluid return line is always submerged beneath dialysis fluid. The straw allows for dialysis fluid to be pulled from the bottom of the recirculation container into the dialysis fluid return line so that the container does not need to be substantially filled in bypass mode to thereafter start recirculation.

One or both container connectors may be quick-connect, e.g., Hansen, connectors enabling fast and easy connections. The lower container connector may also be self-sealing or normally closed, so when the drain line (for example) is disconnected from the recirculation container, the dialysis fluid does not leak from the container. A small container line may be provided and extend from the lower container connector, e.g., for connection to the drain line.

The drain outlet of the recirculation container is provided at a height to enable passive flow to the facility drain when the container fills to the level of the drain outlet. The drain outlet may also be provided with an open/closed valve to enable the reusable recirculation container to operate as a pure collection vessel (to operate without facility drain). A reusable container overflow line extends from the recirculation container and includes a distal drain hook that hooks to the facility drain.

The reusable recirculation container may be provided with a removable, threaded lid such that it is not open to the cleanroom environment during normal operation. A hole may be provided in the lid for receiving the priming line and for possibly receiving complimentary quick connect fittings on the overflow line and the small drain line if provided (e.g., for storage when the recirculation container is not in use). An internal “T” fitting may be placed in fluid communication with the container overflow line, wherein one leg of the “T” fitting connects to a line extending to submersible pump that is powered at the end of production shift to drain residual dialysis fluid to the facility drain before the operator removes the threaded lid, pours out any remaining dialysis fluid and disinfects the inside of the reusable recirculation container with a disinfectant, such as a citric acid solution.

In light of the disclosure set forth herein, and without limiting the disclosure in any way, in a first aspect of the present disclosure, which may be combined with any other aspect, or portion thereof, a dialysis fluid production system includes a water purification unit; at least one concentrate; a dialysis machine configured to receive purified water from the water purification unit and to mix the purified water with the at least one concentrate to form dialysis fluid, the dialysis machine including a drain line and a dialysis fluid return line; a product container positioned and arranged to receive the dialysis fluid from the dialysis machine; and a recirculation container configured to receive an end of the drain line and an end of the dialysis fluid return line, the recirculation container enabling dialysis fluid to be recirculated from the drain line, through the dialysis fluid return line, back to the dialysis machine, the recirculation container further configured to be positioned adjacent to a facility drain such that overflow dialysis fluid flows from the recirculation container into the facility drain.

In a second aspect of the present disclosure, which may be combined with any other aspect, or portion thereof, the dialysis fluid production system includes the facility drain and a cleanroom, the facility drain located within the cleanroom.

In a third aspect of the present disclosure, which may be combined with any other aspect, or portion thereof, the dialysis fluid production system includes a cargo unit configured to be transported by a vehicle, the cleanroom located within the cargo unit.

In a fourth aspect of the present disclosure, which may be combined with any other aspect, or portion thereof, the dialysis machine is configured (i) to recirculate dialysis fluid from the drain line, through the dialysis fluid return line, back to the dialysis machine while the product container is being filled with dialysis fluid and (ii) such that overflow dialysis fluid flows from the recirculation container into the facility drain after the product container has been filled and is being removed.

In a fifth aspect of the present disclosure, which may be combined with any other aspect, or portion thereof, the dialysis fluid production system includes a filter, such as an ultrafilter, located fluidically between the dialysis machine and the product container.

In a sixth aspect of the present disclosure, which may be combined with any other aspect, or portion thereof, the recirculation container is configured to be positioned adjacent to the facility drain such that overflow dialysis fluid flows from the recirculation container into the facility drain by being releasably held within a recirculation fixture, the recirculation fixture configured to rest securely on the facility drain.

In a seventh aspect of the present disclosure, which may be combined with any other aspect, or portion thereof, the recirculation fixture includes a drain mount sized to fit over and rest securely on a top of the facility drain.

In an eighth aspect of the present disclosure, which may be combined with any other aspect, or portion thereof, the recirculation fixture includes a container holder formed so as to hold the recirculation container at a desired angle relative to a horizontal top of the facility drain.

In a ninth aspect of the present disclosure, which may be combined with any other aspect, or portion thereof, the recirculation container includes a spout, and wherein the desired angle sets the spout at about 9° above horizontal.

In a tenth aspect of the present disclosure, which may be combined with any other aspect, or portion thereof, the container holder includes at least one securement feature shaped to contact and orient at least one of the drain line or the dialysis fluid return line.

In an eleventh aspect of the present disclosure, which may be combined with any other aspect, or portion thereof, the recirculation fixture is reusable and the recirculation container is disposable.

In a twelfth aspect of the present disclosure, which may be combined with any other aspect, or portion thereof, the recirculation container includes a closed chamber, which when oriented for operation includes an upper closed chamber line for fluid communication with the facility drain and plural closed chamber lines for fluid communication with the drain line and the dialysis fluid return line.

In a thirteenth aspect of the present disclosure, which may be combined with any other aspect, or portion thereof, the closed chamber is disposable, or wherein the system is configured to run a disinfection sequence in which the closed chamber is disinfected along with the drain line and the dialysis fluid return line.

In a fourteenth aspect of the present disclosure, which may be combined with any other aspect, or portion thereof, the recirculation container includes an inner wall and an outer wall defining a dialysis fluid holding space, the outer wall extending up from the dialysis fluid holding space so as to hold the drain line above dialysis fluid residing within the dialysis fluid holding space, the inner wall forming a weir over which the dialysis fluid overflows from the dialysis fluid holding space into the facility drain.

In a fifteenth aspect of the present disclosure, which may be combined with any other aspect, or portion thereof, the weir additionally forms a mounting hook for removably fixing the recirculation container to the facility drain.

In a sixteenth aspect of the present disclosure, which may be combined with any other aspect, or portion thereof, the recirculation container includes a return tube having an end configured to connect to the dialysis fluid return line.

In a seventeenth aspect of the present disclosure, which may be combined with any other aspect, or portion thereof, the return tube extends from a bottom of the recirculation container.

In an eighteenth aspect of the present disclosure, which may be combined with any other aspect, or portion thereof, the recirculation container includes a connection structure to the facility drain, wherein the connection structure is also where dialysis fluid overflows from the recirculation container into the facility drain.

In a nineteenth aspect of the present disclosure, which may be combined with any other aspect, or portion thereof, the recirculation container includes a vacuum flask, a wash bottle, or plastic piping.

In a twentieth aspect of the present disclosure, which may be combined with any other aspect, or portion thereof, the recirculation container includes a support fixture configured to orient the recirculation container in a desired manner relative to the facility drain.

In a twenty-first aspect of the present disclosure, which may be combined with any other aspect, or portion thereof, the recirculation container includes a beverage dispenser, a squeeze and pour container, or a tip and pour container.

In a twenty-second aspect of the present disclosure, which may be combined with any other aspect, or portion thereof, the recirculation container is configured to be cleaned and reused, the recirculation container including container connectors for fluid communication with the drain line and the dialysis fluid return line, the recirculation container further including a drain outlet for fluid communication with an overflow line configured to extend to the facility drain.

In a twenty-third aspect of the present disclosure, which may be combined with any other aspect, or portion thereof, the container connector for the dialysis fluid return line is located above the container connector for the drain line, and which includes a straw extending from the container connector for the dialysis fluid return line to a bottom of the recirculation container.

In a twenty-fourth aspect of the present disclosure, which may be combined with any other aspect, or portion thereof, the recirculation container includes a container line extending from the container connector for the drain line, the container line configured to connect to the drain line or the dialysis fluid return line.

In a twenty-fifth aspect of the present disclosure, which may be combined with any other aspect, or portion thereof, the recirculation container includes a removable lid, the removable lid including at least one of (i) a hole for receiving a priming line or (ii) at least one complimentary quick connect fitting.

In a twenty-sixth aspect of the present disclosure, which may be combined with any other aspect, or portion thereof, any of the features, functionality and alternatives described in connection with any one or more of FIGS. 1 to 9 may be combined with any of the features, functionality and alternatives described in connection with any other of FIGS. 1 to 9.

In light of the above aspects and disclosure herein, it is accordingly an advantage of the present disclosure to provide a system that makes fresh dialysis fluid on demand when needed, e.g., during a high demand due to disease or other emergency, wherein the system avoids long delays in capacity expansion, validation, etc., by taking advantage of already validated and existing HD machines, equipment and concentrates to produce a final dialysis fluid in an alternative production setting, which is quickly scalable.

It is another advantage of the present disclosure to provide a system that makes fresh dialysis fluid on demand using a single use, disposable fluid path in the drain line and dialysis fluid return line loop, which minimizes the size of a recirculation container, which aids the microbiological cleanliness of both a durable flow path within the hemodialysis (“HD”) machine and the cleanroom in which the HD machine resides, which helps to maintain a contaminant free aseptic filling of product dialysis fluid.

It is a further advantage of the present disclosure to provide a system that makes fresh dialysis fluid on demand, and which minimizes the spillage of dialysis fluid from the recirculation container into the cleanroom.

It is yet another advantage of the present disclosure to provide a system that makes fresh dialysis fluid on demand, and which simplifies the management of the fluid lines including the dialysis fluid return line, the HD machine drain line, the water purification unit drain line, and a priming line, giving each line a dedicated location and minimizing a likelihood of mistake during setup.

It is still another advantage of the present disclosure to provide a system that makes fresh dialysis fluid on demand using a disposable recirculation container that may be used as-purchased, without modification.

It is yet a further another advantage of the present disclosure to provide a system that makes fresh dialysis fluid on demand, and which enables use of a relatively inexpensive and small volume recirculation container.

It is still a further another advantage of the present disclosure to provide a system that makes fresh dialysis fluid on demand, and which empties dialysis fluid into the recirculation container during bypass when product dialysis fluid flow is stopped to switch bags on a filling line with no monitoring or intervention required from the operator.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

FIG. 1 is a schematic view of one embodiment for a mobile emergency dialysis fluid generation system of the present disclosure.

FIG. 2 is a schematic flowpath for one embodiment of the dialysis machine of the present disclosure.

FIG. 3 is a perspective view of the system using a first primary recirculation container embodiment of the present disclosure.

FIGS. 4A and 4B are elevation views of the first primary recirculation container embodiment of the present disclosure.

FIGS. 5A to 5C are perspective views of alternative recirculation container embodiments of the present disclosure, which operate with support fixtures.

FIGS. 6A to 6C are perspective views of the system using a second primary recirculation container embodiment of the present disclosure.

FIG. 7 is a perspective view of the system using a third primary recirculation container embodiment of the present disclosure.

FIGS. 8A to 8C are perspective views of alternative recirculation container embodiments of the present disclosure, which include common facility drain connection and dialysis fluid overflow structure.

FIG. 9 a perspective view of the system using a fourth primary recirculation container embodiment of the present disclosure.

### DETAILED DESCRIPTION

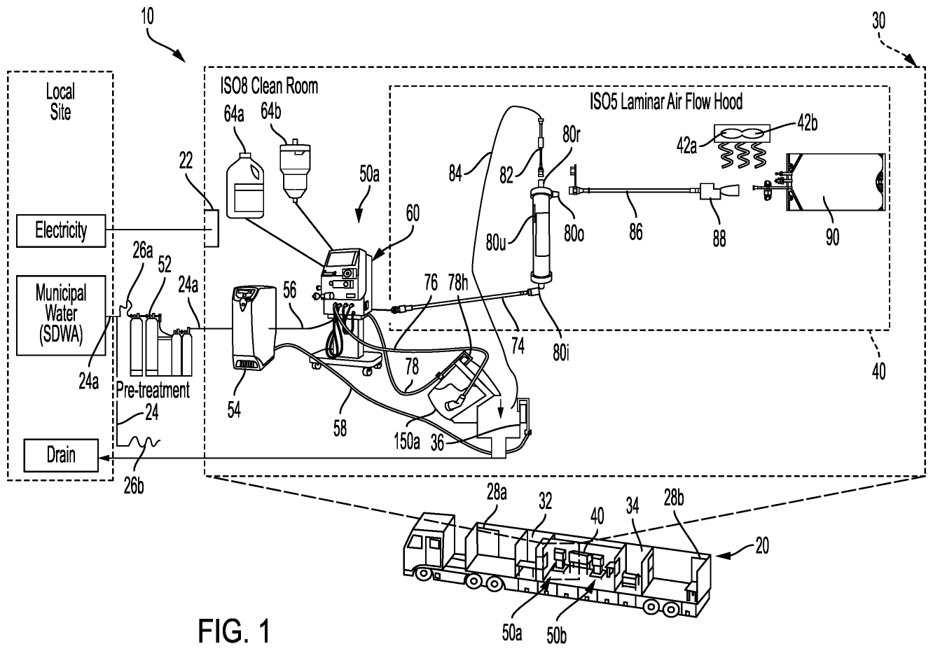

Referring now to the drawings and in particular to FIG. 1, an embodiment of a mobile dialysis fluid generation system is illustrated by system 10. FIG. 1 illustrates that system 10 includes a cargo unit 20. Cargo unit 20 may be constructed of metal and be configured to be loaded onto a flatbed truck, railcar or other transportation medium for moving from one hospital or clinic to another, or for moving to or from a location at which cargo unit 20 of system 10 may be restocked with the raw materials discussed herein. Cargo unit 20 in the illustrated embodiment includes an electrical panel 22, e.g., fuse box, which accepts outside electrical power from a local site, e.g., 110 to 130 VAC (for United States) and 220 to 240 VAC (for United States or Europe), and which may include one or more transformer for producing any desired AC or DC for the electrical components of system 10.

Electrical panel 22 in one embodiment powers a plurality of lights (not illustrated) located inside cargo unit 20 of system 10. In addition to controlling cleanroom air through purification and laminar flow, controlling operator contamination through gowning, and performing manual cleaning procedures, the cleanliness of system 10 may be aided by providing at least some of the lights as ultraviolet (“UV”) lights that tend to disinfect or purify the air outside of cleanroom 30. In this manner the operator(s) reside in a semi-clean environment prior to entering cleanroom 30. Alternatively or additionally, electrical panel 22 may power disinfectant sprayers (not illustrated) that periodically spray disinfectant into the air outside of cleanroom 30. Here again, the disinfectant spray allows the operator(s) to reside in a semi-clean environment prior to entering cleanroom 30.

Cargo unit 20 as illustrated in FIG. 1 may also include plumbing 24, which includes inlet end 24a configured to connect to an external water supply (e.g., municipal water supply) brought to cargo unit 20. Plumbing 24 also includes connection ends configured to connect to hard or flexible water hoses 26a, 26b. A hard or flexible water hose 26a, 26b is provided for each dialysis fluid preparation unit 50a, 50b provided within cargo unit 20. In the version of system 10 discussed herein, two dialysis fluid preparation units 50a, 50b are illustrated and described, however, a single or three or more dialysis fluid preparation units 50a . . . 50n may be provided instead. Cargo unit 20 also includes one or more doors 28a and 28b, which allow operator entry into and exit out of the cargo unit.

Cargo unit 20 includes or holds cleanroom 30, which is able to be placed in any desired location within cargo unit 20. In one example, a frame defining cleanroom 30 also defines or provides a finished goods area 32 and a gowning and de-gowning area 34. For example, finished goods area 32 and gowning and de-gowning area 34 may be provided with exterior doors that allow entrance and exit from the overall frame forming cleanroom 30. Interior doors may be provided from cleanroom 30 into/out of finished goods area 32 and from cleanroom 30 into/out of gowning and de-gowning area 34.

In the illustrated embodiment of FIG. 1, product movement within cargo unit 20 is generally from right to left, where raw materials and supplies are provided to the right of de-gowning area 34, while finished products, e.g., bagged dialysis fluid, are stored to the left of finished goods area 32. Production operator(s) may enter cargo unit 20 through rightmost door 28b, obtain needed raw materials, enter gowning and de-gowning area 34 via an exterior door, don the necessary gowns, enter cleanroom 30 via an interior door, operate the equipment discussed herein, place finished goods in finished goods area 32 through an interior door, and after a shift enter gowning and de-gowning area 34 through an interior door, remove their gown(s), and exit gowning and de-gowning area 34 via the exterior door. The finished products may be pulled by another operator(s) from finished goods area 32 and be stored to the left of finished goods area 32. The finished goods operator(s) may enter cargo unit 20 via leftmost door 28a. At the end of the day, the operators exit the door they came in through in one embodiment. In one preferred embodiment, raw materials are transferred in a bolus into cleanroom 30 at the start of a production shift, and finished containers or bags are transferred out of the cleanroom as they are produced.

Cleanroom 30 in the illustrated embodiment includes and holds a laminar flow hood 40, which may be an ISO5 laminar flow hood, and which may be located between dialysis fluid preparation units 50a and 50b. As described in detail below, dialysis fluid preparation units 50a, 50b each output dialysis fluid to an area located within laminar flow hood 40. It is accordingly desirable in one embodiment to locate laminar flow hood 40 centrally amongst dialysis fluid preparation units 50a . . . 50n.

In an alternative embodiment, it is contemplated to provide cleanroom 30 including finished goods area 32 and gowning and de-gowning area 34 as a modular “pop up” structure, which is placed in a desired location other than cargo unit 20. System 10 may alternatively or additionally provide cleanroom 30 including finished goods area 32 and gowning and de-gowning area 34 as a standalone unit in a desired location, such as (i) in a convention space, warehouse, auditorium, vacant retail or other suitably large space located near the hospital or clinic, (ii) in a mobile office space placed near or in proximity to the hospital or clinic, (iii) in the hospital or clinic itself, or (iv) in a 503b compounding facility. Cleanroom 30 in an embodiment is built and qualified in a space controlled by the assignee of the present disclosure. It should be appreciated that regardless of whether cleanroom 30 is located in cargo unit 20 or as a modular “popup” cleanroom at any of the other locations listed above, the cleanroom includes any and all structure, functionality and alternatives discussed herein.

Laminar flow hood 40 in the illustrated embodiment includes laminar flow fans 42a, 42b. While two laminar flow fans 42a, 42b are illustrated, a single fan or three or more laminar flow fans 42a . . . 42n may be provided alternatively. Laminar flow fans 42a and 42b are configured to blow cleanroom or high-efficiency particulate air (“HEPA”) air at a laminar flowrate, e.g., 90 feet/minute (0.46 meters/second), across an area within hood 40.

Dialysis fluid preparation units 50a, 50b each include a water pretreatment subsystem 52, e.g., located outside cleanroom 30, and connected to one of hard or flexible water hoses 26a, 26b. Each water pretreatment subsystem 52 may include, for example, any one or more of carbon filtration, a chlorine remover, particulate filter(s), a water softener, and/or an inline ultraviolet (“UV”) disinfection device. The water pretreatment subsystems 52 output to water purification units 54. Water purification units 54 may include and one or more type of water purification and associated structures, including reverse osmosis (“RO”), ultraviolet (“UV”) radiation, electrodeionization, ultrafiltration, ion-exchange resins, and/or heat disinfection. One suitable water purification unit 54 is provided by the assignee of the present disclosure and may be marketed under the product name WRO300H. The water pretreatment subsystems 52 and water purification units 54 may each be individually or collectively referred to herein as water purification equipment. It is intended that water exiting water purifier 54 be of a purification quality suitable for use in a hemodialysis treatment, such as a continuous renal replacement therapy (“CRRT”).

Water purification units 54 of dialysis fluid preparation units 50a, 50b each output to a dialysis machine 60 configured to receive purified water from the water purification equipment and to mix the purified water with the at least one concentrate to form dialysis fluid. One suitable dialysis machine for system 10 is an AK 98™ Dialysis Machine marketed by the assignee of the present disclosure. The use of existing dialysis machine 60 enables system 10 to be assembled relatively quickly using proven mixing technology and without having to develop and validate a dedicated mixing device and associated process.

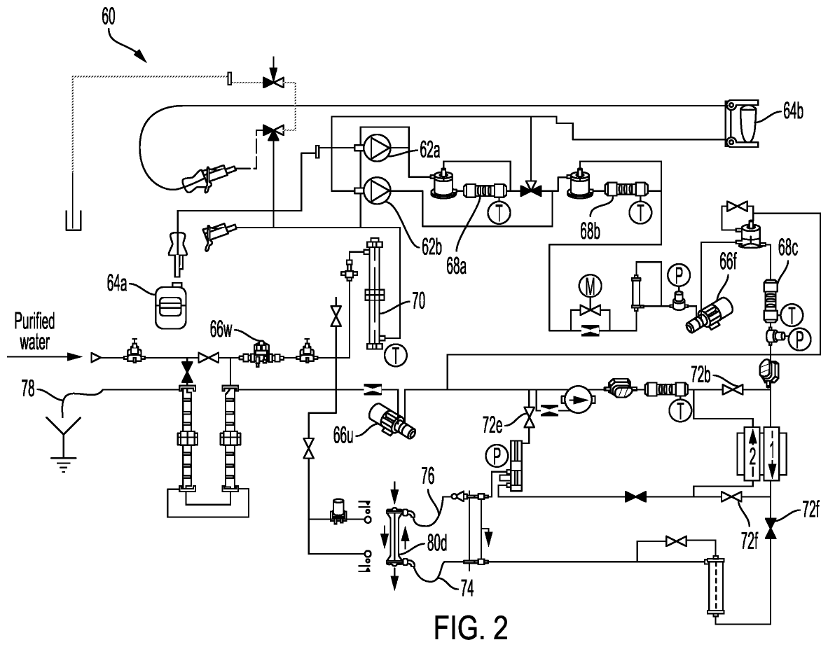

As illustrated additionally in FIG. 2, dialysis machine 60 includes at least one mixing pump 62a, 62b for mixing purified water from water purification unit 54 with at least one concentrate 64a, 64b. Dialysis machine 60 in one embodiment includes a purified water pump 66w that pulls purified water from water purification unit 54, such that water purification unit 54 may, but does not have to have, its own water pump. In an alternative embodiment, water purification unit 54 includes a pump that pumps purified water under positive pressure to dialysis machine 60. Here, dialysis machine 60 does not have to have a pump to pump purified water from water purification unit 54.

As illustrated in FIG. 2, dialysis machine 60 includes B-concentrate pump 62b for metering purified water through bicarbonate cartridge 64b and A-concentrate pump 62a for metering liquid acid concentrate from concentrate container 64a into the mixture of purified water and bicarbonate concentrate. Temperature compensated conductivity cells 68a, 68b, 68c are used in one embodiment to ensure the proper bicarbonate concentrate mixture with purified water and the proper mixture of bicarbonate concentrate and purified water with acid concentrate.

Dialysis machine 60 may also include a heater 70, such as an inline heater. Heater 70 may or may not be energized during the preparation of the dialysis fluid but is used after a production shift for disinfecting dialysis machine 60 and the lines associated with dialysis fluid preparation unit 50a, 50b. Dialysis machine 60 includes a fresh dialysis fluid pump 66f for delivering fresh (possibly heated) dialysis fluid to a dialyzer 80d for treating a patient. Dialysis machine 60 includes a used dialysis fluid pump 66u for removing used dialysis fluid from dialyzer 80d after treating a patient.

FIGS. 1 and 2 also illustrate the disposable portion of dialysis fluid preparation units 50a, 50b. The disposable portion of dialysis fluid preparation units 50a, 50b includes a fresh dialysis fluid line 74 and a dialysis fluid return line 76. Each of lines 74 and 76 may be a flexible tube. FIG. 2 illustrates a dialysis machine 60 in a hemodialysis treatment setting in which fresh dialysis fluid line 74 is connected to an inlet of dialyzer 80d, while dialysis fluid return line 76 returns used dialysis fluid from dialyzer 80d to the used dialysis fluid circuit, including used dialysis fluid pump 66u. FIG. 2 further illustrates that in a normal treatment operation, dialysis machine 60 pumps used dialysis fluid through a flexible drain line 78 to house drain at the clinic or hospital.

FIG. 1 illustrates how the flexible lines of dialysis machine 60 are configured (most reconfigured) for use with fluid preparation units 50a, 50b. Here, fresh dialysis fluid line 74 extends to an inlet 80i of an ultrafilter 80u for further filtering of the fresh dialysis fluid so that it is of a quality suitable for use in an HD (CRRT) treatment. Dialysis fluid return line 76 instead of being connected to dialyzer 80d, is in FIG. 1 routed instead to a recirculation container 150a as discussed in detail herein. Drain line 78 is likewise routed to recirculation container 150a and in the illustrated embodiment includes a drain hook 78h that hooks over an edge of the recirculation container.

Recirculation container 150a as discussed in detail herein operates with a facility drain 36 provided within cleanroom 30 of cargo unit 40. FIG. 1 further illustrates a purified water line 56 carrying purified water from water purification unit 54 to dialysis machine 60. A water purification unit drain line 58 is also provided and extends from water purification unit 54 to facility drain 36.

Regarding ultrafilter 80u, an adapter 82 is connected to a deaeration port 80r of the ultrafilter. A priming line 84 extends from adapter 82 to facility drain 36. Priming line 84 enables ultrafilter 80u to be fully primed with dialysis fluid at the beginning of a product shift. A product line 86 carries filtered or product dialysis fluid from an outlet port 80o of ultrafilter 80u, via an adapter 88, to a product container 90, e.g., a presterilized dialysis fluid bag.

A separate product container or bag 90 is used for each fill, so if a filling session includes two-hundred fills, then two-hundred containers 90 are consumed. Containers 90 may be made of any of the materials discussed above. Containers 90 in an embodiment are provided preprinted or pre-labeled and presterilized. Alternatively, labels are added. Containers 90 are provided to hold a desired amount of dialysis fluid, such as one, two, four, five or six liters.

Any or all of flexible lines 56, 58, 74, 76, 78, 84 and 86, adapters 82, 88, ultrafilter 80u, product container 90, and any of the recirculation containers discussed herein, including container 150a, may be made of one or more plastic, e.g., ethylene vinyl acetate (“EVA”), polyvinylchloride (“PVC”) or a non-PVC material, such as polyethylene (“PE”), polyurethane (“PU”) or polycarbonate (“PC”). A tubing set including ultrafilter 80u, adapter 82, priming line 84, product line 86 and adapter 88 is used for a single product shift, e.g., for one day or for the time it takes to make a predefined number of filled dialysis fluid containers 90. After this time or production number, the tubing set is discarded and replaced.

In an alternative embodiment, fresh dialysis fluid instead of being pumped through ultrafilter 80u (e.g., if the preferred ultrafilters are unavailable) is pumped into the blood side of dialyzer 80d, through the very small pores of the hollow fiber membranes of the dialyzer, up or down through the outsides of the hollow fiber membranes and out of the dialysis fluid side of dialyzer 80d at the other end of the dialyzer. The used dialysis fluid port of dialyzer 80d is capped to force the above-described filtration.

Referring again to FIGS. 1 and 2, in normal operation, the dialysis machine 60 delivers fresh dialysis fluid to dialyzer 80d and removes used dialysis fluid from the dialyzer. The used dialysis fluid is delivered to drain via flexible drain line 78. It is common across multiple manufacturers of dialysis machines to require the used dialysis fluid pump to pump used dialysis fluid from the dialyzer to drain in order for the fresh dialysis fluid pump to pump fresh dialysis fluid to the dialyzer. So for the present dialysis fluid preparation units 50a, 50b, it is not possible (without substantial modification to the software, which would have to be validated) to only run fresh dialysis fluid pump 66f, concentrate pumps 62a, 62b and purified water pump 66w without running the used dialysis fluid pump 66u. Used dialysis fluid pump 66u needs to be operated as well.

To enable used dialysis fluid pump 66u to be operated during operation of system 10, the system provides a recirculation container 150a (and others described below) that receives both the distal end of drain line 78 and the end of dialysis fluid return line 76, which is normally connected to dialyzer 80d. Drain line 78 in one embodiment terminates with drain hook 78h, which hooks over the side wall of recirculation container 150a so that dialysis fluid is introduced above a level of dialysis fluid within recirculation container 150a. Dialysis fluid return line 76 in one embodiment extends to a bottom of recirculation container 150a so that a negative pressure can be applied to pull dialysis fluid from recirculation container 150a, along dialysis fluid return line 76, to dialysis machine 60.

At the startup of a production shift, each dialysis machine 60 is primed with dialysis fluid. The entire dialysis fluid circuit including the water lines, the concentrate lines, the fresh dialysis fluid lines, and the used dialysis fluid lines are primed. Flexible fresh dialysis fluid line 74 and ultrafilter 80u are primed via priming line 84. Additionally, flexible dialysis fluid return line 76 and drain line 78 are primed, leaving a volume of dialysis fluid within recirculation container 150a.

During the production of dialysis fluid, as dialysis fluid is filling a presterilized product container 90, used dialysis fluid pump 66u pulls the priming dialysis fluid volume from recirculation container 150a into dialysis machine 60 via dialysis fluid return line 74 and pushes the dialysis fluid from dialysis machine 60 to recirculation container 150a via drain line 78. In this manner, the priming dialysis fluid is recirculated in a closed loop between recirculation container 150a and dialysis machine 60 as a product container 90 is being filled with freshly prepared dialysis fluid.

When container or bag 90 is full of dialysis fluid and needs to be removed and replaced with a new empty presterilized product container 90, the operator presses a bypass button on dialysis machine 60. Pressing of the bypass button causes the valve state within the dialysis machine to switch, namely, for fresh dialysis fluid valves 72f and evacuation valve 72e to close and for bypass valve 72b top open. The bypass valve state causes newly prepared dialysis fluid to be diverted to drain via drain line 78 instead of flowing to ultrafilter 80f. Also, because used dialysis fluid pump 66u needs to be operated to divert the newly prepared dialysis fluid to drain, used dialysis fluid pump 66u can no longer be used to pull dialysis fluid along dialysis fluid return line 76 from recirculation container 150a to dialysis machine 60. The bypass valve state accordingly creates a situation in which dialysis fluid flows into recirculation container 150a via the drain line but does not flow back to dialysis machine 60 via dialysis fluid return line 76. The level of dialysis fluid in recirculation container 150a increases accordingly.

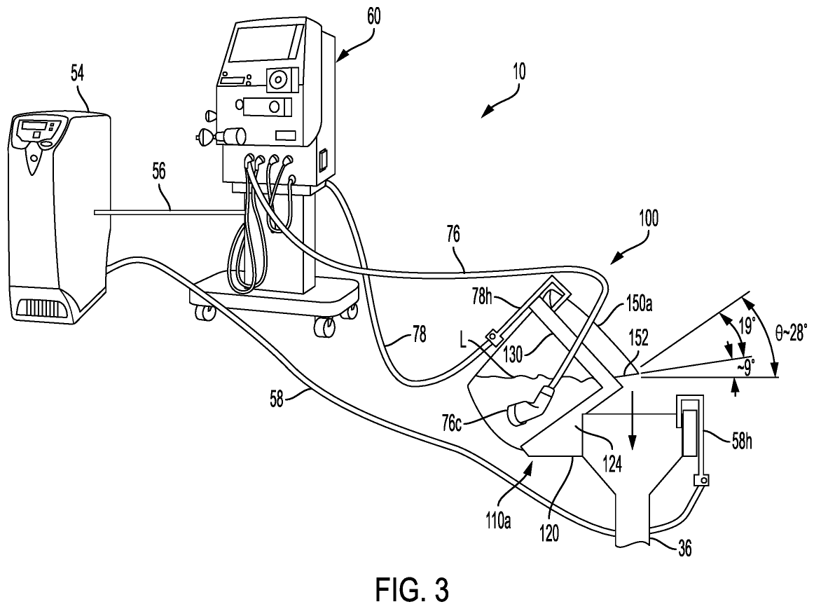

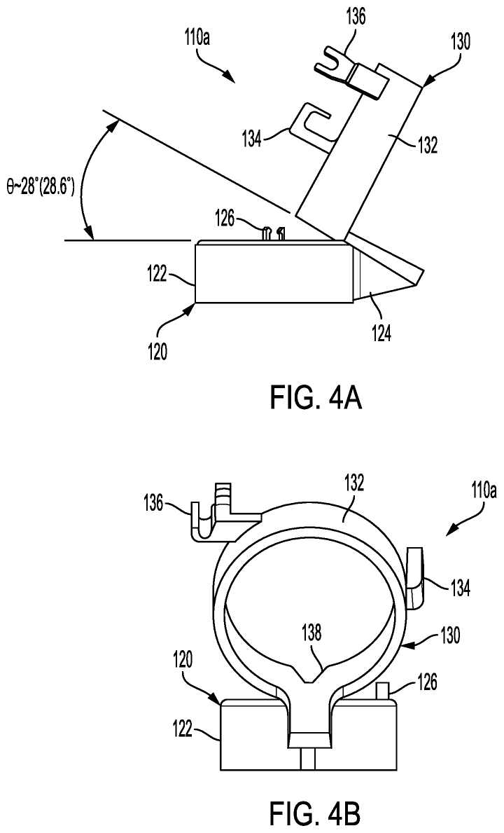

Referring now to FIGS. 3, 4A and 4B, a first primary embodiment for the recirculation containers of the present disclosure includes recirculation container 150a. Here, recirculation container 150a is a beaker, e.g., plastic or glass, which is provided as part of a recirculation assembly 100 along with a recirculation fixture 110a. Recirculation fixture 110a, which may be made of any of the materials discussed herein, does not touch fluid and is a durable component that is reused over multiple production shifts. Recirculation container 150a does touch dialysis fluid as discussed in detail and is a disposable component that is discarded after each shift. Recirculation containers 150a may be packaged in sealed pouches and then sterilized for use in cleanroom 30.

Recirculation fixture 100 is plastic in one embodiment and may be molded or made using an additive manufacturing process. Recirculation fixture 110a includes a drain mount 120 sized to fit over and rest securely on the top of facility drain 36. Drain mount 120 may be in the form of a flanged ring 122 sized to fit around, e.g., snugly around, the largest outer diameter of facility drain 36. A container holder 130 extends from, e.g., is formed with, drain mount 120. Container holder 130 may likewise have a cylindrical or ring shape 132 sized to frictionally receive recirculation container 150a; and/or recirculation container 150a may be provided with a lip extending around the open end of the container, wherein the lip rests against container holder 130 so as to hold recirculation container 150a in a set position.

As illustrated best in FIGS. 3 and 4A, container holder 130 extends at an angle [symbol] relative to drain mount 120, wherein the angle [symbol] is selected so as to allow a volume of dialysis fluid (having a fluid level L) to be held within recirculation container 150a that is sufficient to fully submerge the connector end 76c (e.g., female Hansen connector) of dialysis fluid return line 76, while filtered dialysis fluid is filling a product container 90 and used dialysis fluid pump 66u is recirculating dialysis fluid from recirculation container 150a back to dialysis machine 60. Angle [symbol] is also selected so that during the bypass valve state when dialysis fluid accumulates in recirculation container 150a, the dialysis fluid overflows cleanly from a spout 152 of recirculation container 150a into facility drain 36 during an overflow situation, while preventing dialysis fluid drops from forming and flowing along the outside of recirculation container 150a, e.g., in a non-overflow or static situation, which could create a contamination risk.

The angle [symbol] between container holder 130 and the drain mount 120 is dependent on the angle of spout 152, which in the illustrated embodiment of FIG. 3 is about 19° from the side of recirculation container or flask 150a. It has been found that holding recirculation container such that its spout extends about 9° above horizontal as illustrated in FIG. 3 provides sufficient dialysis fluid volume accumulation during dialysis fluid recirculation (to submerge the connector end 76c of dialysis fluid return line 76), while allowing the overflow dialysis fluid to flow cleanly into facility drain 36 during the standby valve state (while bags 90 are swapped). In one specific example using a beaker manufactured by Globe Scientific, part #BPM1000P having the 19° spout, an angle of around 28° (e.g.,) 28.6° between container holder 130 and the drain mount 120 causes the container holder to hold the beaker such that the bottom of spout 152 is roughly 9° degrees above horizontal. It should be appreciated that different recirculation containers with different spouts 152 may require different [symbol] angles between container holder 130 and drain mount 120, however, it is believed that an angle [symbol] between and including 20° to 40° should work with virtually all recirculation containers (e.g., beakers) of the first primary embodiment. In the illustrated embodiment, a gusset 124 is provided (e.g., molded with) between container holder 130 and drain mount 120 for rigidity and to produce a more robust recirculation fixture 110a.

The size of recirculation container 150a may be on the order of one liter or less, where smaller containers are less expensive and easier to stock, but where return line 76 becomes difficult to properly immerse if recirculation container 150a is too small. Ring 132 of container holder 130 is sized to hold recirculation container 150a firmly throughout a production shift, but to allow recirculation container 150a to be removed easily at the end of the shift, e.g., by sliding out from ring 132 of container holder 130. Ring 132 may be cylindrical or slightly conical if recirculation container 150a is likewise conical. In FIG. 3, recirculation container 150a slides into ring 132 from the upper right and out from ring 132 in the opposite direction. In FIG. 4A, recirculation container 150a slides into ring 132 from the upper left and out from ring 132 in the opposite direction. In FIG. 4B, recirculation container 150a slides into ring 132 from behind and out from ring 132 in the opposite direction.

One or both of container holder 130 and drain mount 120 of recirculation fixture 110a may be provided with securement features for contacting and orienting the various lines running to recirculation container 150a and to facility drain 36. In the illustrated embodiment, container holder 130 includes a first securement feature 134 for contacting and orienting dialysis fluid return line 76 and a second securement feature 136 for contacting and orienting drain hook 78h of drain line 78, which each extend into recirculation container 150a. In the illustrated embodiment, securement feature 134 holds dialysis fluid return line 76 so that its connector end 76c is biased to extend to the bottom of recirculation container 150a. Securement feature 136 holds drain hook 78h of drain line 78 such that an outlet of drain hook 78h resides above the level L of dialysis fluid within recirculation container 150a during recirculation and during the bypass valve state overflow.

Securement features 134 and 136 are provided on the outside of ring 132 so as not to interfere with the insertion and removal of recirculation container 150a. FIG. 4B also illustrates that a groove 138, e.g., V-shaped groove is formed in ring 132 to accept and properly orient spout 152 of recirculation container 150a. Groove 138 helps to prevent recirculation container 150a from turning when routing various lines through securement features 134 and 136.

Drain mount 120 of recirculation fixture 110a may also include one or more securement feature. For example, drain mount 120 may include a first securement feature 126 for contacting and orienting priming line 84, which extends from deaeration port 80r of ultrafilter 80 into facility drain 36. A second securement feature (not illustrated) may be provided to contact and orient water purification unit drain line 58 that extends from water purification unit 54 into facility drain 36. In the illustrated embodiment the distal end of water purification unit drain line 58 is provided with a drain line hook 58h, which is hooked over the side of facility drain 36. Priming line 84 and water purification unit drain line 58 both extend to facility drain 36 rather than recirculation container 150a, so their associated securement features if provided are formed with drain mount 120, which rests atop the facility drain 36.

A separate facility drain 36 may be provided for each dialysis fluid preparation unit 50a, 50b. Or, a single facility drain 36 may be provided for multiple dialysis fluid production units 50a, 50b. In such a case, recirculation fixture 110a may include multiple container holders 130 molded in place with a single drain mount 120. Here, drain mount 120 may include securement features 126 for multiple priming lines and multiple water purification unit drain lines. The multiple container holders 130 each hold a recirculation container 150a for a dedicated dialysis machine 60 and are provided with securement features 134, 136 for dialysis fluid return line 76 and drain hook 78h of drain line 78, respectively, as discussed.

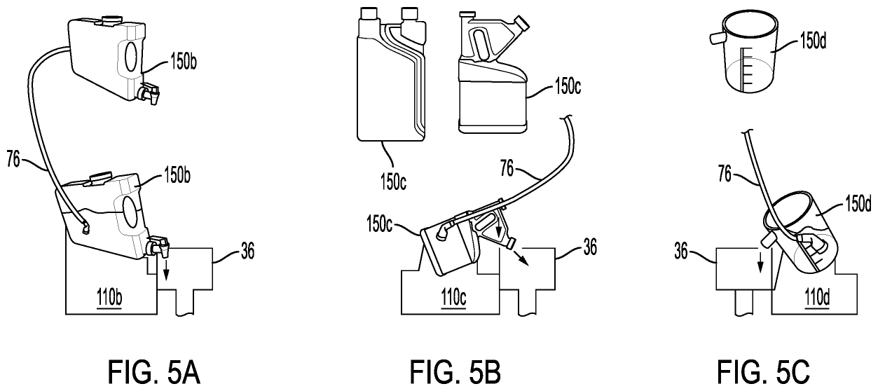

Referring now to FIGS. 5A to 5C, it should be appreciated that recirculation fixture 110a may be configured differently to hold recirculation containers having different shapes than beaker 150a just described. The recirculation containers may instead be more closed and provide multiple openings for dialysis machine drain line 78 and dialysis fluid return line 76. Examples of such containers are illustrated in FIGS. 5A and 5B and include beverage dispensers 150b, squeeze and pour containers and tip and pour containers 150c. Such containers may operate with dedicated and optimized support fixtures 110b and 110c, respectively, for orienting respective containers 150b or 150c in a desired manner relative to facility drain 36. FIG. 5C illustrates an arrangement similar to recirculation fixture 110a and recirculation container 150a, in which a further modified and optimized recirculation fixture 110d holds an overflow can 150d having a cylindrical spout 152 in a desired orientation relative to facility drain 36.

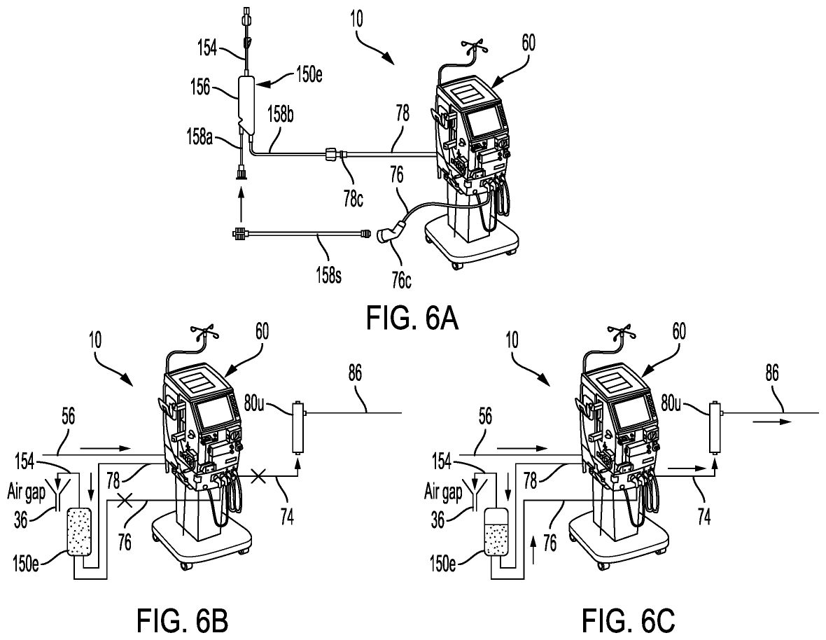

Referring now to FIGS. 6A to 6C, in a second primary and alternative embodiment, an alternative recirculation container 150e is instead a closed chamber, which may be a disposable part formed from a blow or injection molding process, for example, an existing HD expansion chamber accessory provided by the assignee of the present disclosure. Recirculation container 150e may be made from any of the materials discussed herein. When oriented for operation in a generally vertically extending configuration, closed chamber 150c includes an upper closed chamber line 154 extending vertically upward from a top of a closed chamber body 156 and two lower closed chamber lines 158a and 158b extending vertically downward from a bottom of closed chamber body 156.

With closed chamber body 156 fixed in a generally vertical orientation to a side of facility drain 36, upper closed chamber line 154 may be bent 180° and extended into the facility drain 36, e.g., so as to leave an air gap between the end of upper closed chamber line 154 and the dialysis fluid level within facility drain 36 (FIGS. 6B and 6C). Lower closed chamber line 158b is connected and sealed fluidically to dialysis machine drain line 78, e.g., via a hose clamp 78c that in effect replaces drain line hook 78h. The other lower closed chamber line 158a is connected and sealed fluidically to dialysis fluid return line 76, e.g., via an intermediate line segment 158s that includes the correct counterpart connectors for closed chamber line 158a and dialysis fluid return line 76 (e.g., mating Hansen connector).

FIG. 6B illustrates the situation in which system 10 is in the bypass valve state so that the operator can remove a filled dialysis fluid container 90 and replace it with an empty presterilized dialysis fluid container 90. Here, no dialysis fluid flow occurs in dialysis fluid return line 76 or through ultrafilter 80u via lines 74 and 86. Newly prepared fresh dialysis fluid is instead diverted to closed chamber 150e, which fills completely and overflows via upper closed chamber line 154 into facility drain 36. FIG. 6C illustrates the situation in which system 10 is filling dialysis fluid container 90 with fresh, filtered dialysis fluid. Here, return dialysis fluid flows from closed chamber 150e via dialysis fluid return line 76 to dialysis machine 60 and is recirculated back to closed chamber 150e via drain line. Newly prepared dialysis fluid flows through ultrafilter 80u via lines 74 and 86 to dialysis fluid container 90.

Closed chamber body 156 has a small volume in one embodiment, which remains at least partially full during dialysis fluid recirculation between closed chamber body 156 and dialysis machine 60. Closed chamber 150e may be provided in a presterilized package and be discarded at the end of a production shift. Due to the small volume and enclosed nature of closed chamber body 156, however, it may be possible to instead disinfect closed chamber 150e, including its lines, along with dialysis machine 60 at the end of treatment. In this manner, the same closed chamber 150e may be employed as the dialysis fluid recirculation container over multiple production shifts.

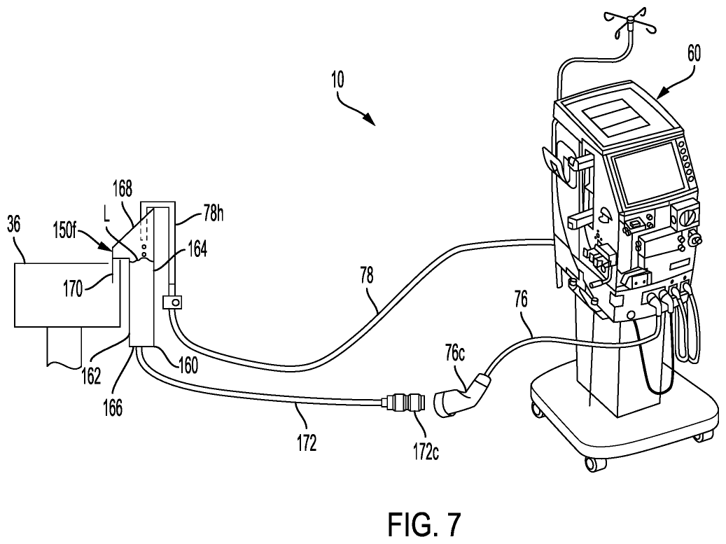

Referring now to FIG. 7, in a third primary and alternative embodiment, an alternative recirculation container 150f is configured to also allow it to be secured to facility drain 36. Recirculation container 150f may be made from any of the materials discussed herein. So like with the second primary embodiment (closed chamber 150e), the durable or reusable recirculation fixture 110a of the first primary embodiment is removed. In the third primary embodiment, recirculation container 150f includes a body 160 defining a dialysis fluid holding space. Body 160 includes an inner wall 162, an outer wall 164, a bottom wall 166 and sidewalls 168. Inner wall 162 abuts up against the outer diameter of facility drain 36. Outer wall 164 is set back from inner wall 162 a distance sufficient to create, along with the depth of the container, a desired volume for a dialysis fluid holding space within body 160. The inner and outer walls 162 and 164 may be curved to follow the outer diameter of facility drain 36. The inner and outer walls 162 and 164 may also be sized to allow multiple recirculation containers 150f, for multiple dialysis machines 60 and dialysis fluid preparation units 50a, 50b, to be fitted to the same facility drain 36 at once.

Outer wall 164 and sidewalls 168 in one embodiment extend up from the dialysis fluid holding space a distance sufficient to prevent dialysis fluid from spilling over those walls and for outer wall 164 to hold drain hook 78h of dialysis fluid drain line 78, so that dialysis fluid exits drain hook 78 above the dialysis fluid level L. Outer wall 164 may be provided with a notch for holding drain hook 78h so that it cannot slide along the top of the outer wall. Inner wall 162 forms a weir or dam structure 170, which (i) provides a mounting hook that enables recirculation container 150f to be removably fixed to facility drain 36 and (ii) is the vertically lowest wall of container 150f when mounted such that dialysis fluid overflows out of recirculation container 150f, over inner wall 162 and weir 170, and into facility drain 36. Weir 170 is in one embodiment curved along with inner wall 162 to follow the outer diameter of facility drain.

A return tube 172 extends from bottom 166 of body 160 and is fitted at its end with a connector 172c, e.g., male Hansen connector, which is configured to connect to connector 76c, e.g., female Hansen connector, located at the end of dialysis fluid return line 76. Here, connector 76c at the end of dialysis fluid return line 76 does not have to be submerged in recirculation container 150f, and it is ensured that the intake to the dialysis fluid return line 76 is at the lowest point of body 160 of recirculation container 150f. During recirculation, dialysis fluid flows between recirculation container 150f and dialysis machine 60, while a constant level L of dialysis fluid is maintained within the dialysis fluid holding space of body 160. When dialysis machine 60 is in the bypass valve state, the level L of dialysis fluid rises and eventually overflows over inner wall 162 and weir 170 into facility drain 36.

Other than the notch in the outer wall, recirculation container 150f of the third primary embodiment does not need securement features, such as for dialysis fluid return 76 line since it is attached to return tube 172 of recirculation container 150f. Recirculation container 150f may nevertheless provide one or more securement feature for priming line 84 and water purification unit drain line 58, which would be held by the fixtures and extend past recirculation container 150f and into facility drain 36. Recirculation container 150f of the third primary embodiment is in one embodiment packaged in a presterilized pouch and discarded after a product shift.

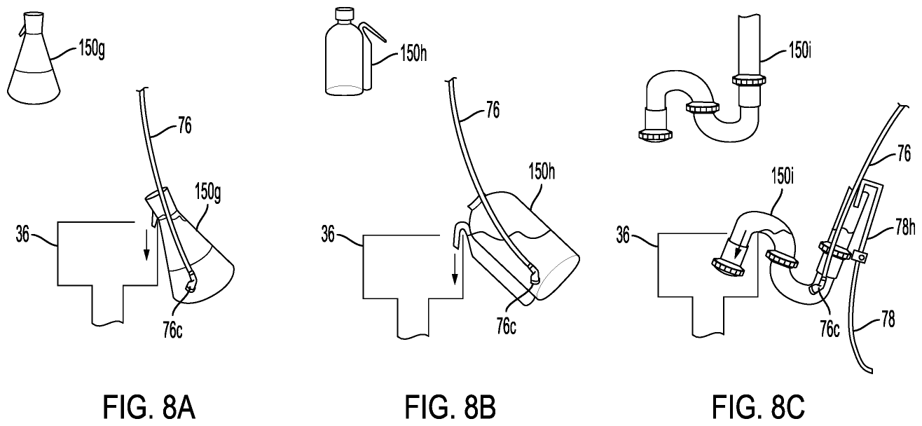

In addition to weir recirculation container 150f just described, FIGS. 8A to 8C illustrate other recirculation containers in which the connection structure to facility drain 36 also serves as the place where overflow dialysis fluid flows into the facility drain. Such alternative recirculation containers may include a vacuum flask 150g as illustrated in FIG. 8A, a wash bottle 150h as illustrated in FIG. 8B, and plastic piping 150i as illustrated in FIG. 8C. Unlike recirculation container 150f, connector end 76c of dialysis fluid return line 76 is submerged into alternative recirculation containers 150g, 150h, 150i.

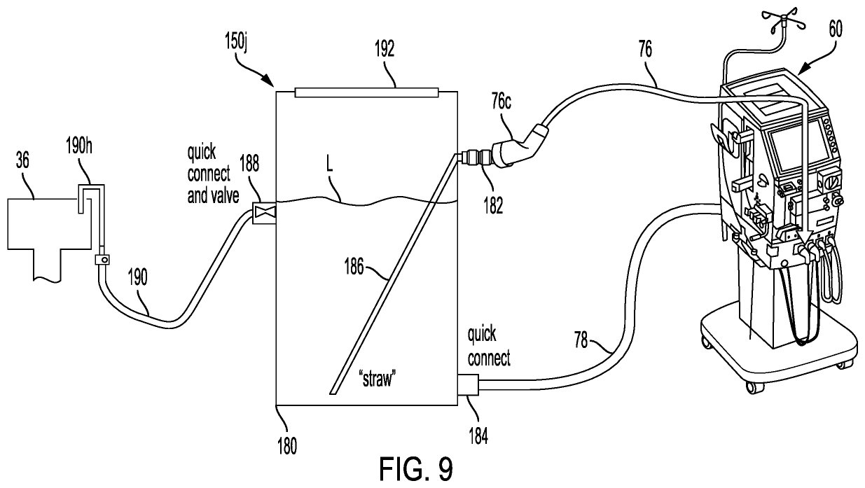

Referring now to FIG. 9, in a fourth primary and alternative embodiment, recirculation container 150j instead includes a larger, durable recirculation tank 180 that is reused and periodically cleaned, e.g., after each production shift. In one embodiment, the dialysis machine drain line 78 extends to an upper container connector 182, while the dialysis fluid return line extends to a lower container connector 184 such that dialysis fluid enters the container above a level L of dialysis fluid and so that there is plenty of head of dialysis fluid to feed dialysis fluid return line 76. In an alternative embodiment and as illustrated in FIG. 9, say if the upper container connector 182 is a matching connector for connector 76c of dialysis fluid return line 76 (e.g., Hansen connection), then lines 76 and 78 may be reversed with the dialysis fluid return line 76 connecting to upper container connector 182 and dialysis machine drain line 78 connecting to the lower container connector 184. Here, a straw 186 extending from upper container connector 182 to the bottom of tank 180 may be provided inside of the tank so that the inlet to the dialysis fluid return line 76 is always submerged beneath dialysis fluid. Straw 186 allows for dialysis fluid to be pulled from the bottom of tank 180 of recirculation container 150j into dialysis fluid return line 76 so that tank 180 does not need to be substantially filled in bypass mode to thereafter start the recirculation.

One or both container connectors 182, 184 may be quick-connect, e.g., Hansen, connectors enabling fast and easy connections. Lower container connector 184 may also be self-sealing so when drain line 78 (for example) is disconnected from recirculation container 150j, dialysis fluid does not leak from the container. A small container line (not illustrated) may be provided and extend from the lower container connector 184, e.g., for connection to drain line 78.

A drain outlet 188 of recirculation container 150j is provided at a height to enable passive flow to facility drain 36 when tank 180 container fills to the level of drain outlet 188. Drain outlet 188 may also be provided with an open/closed valve to enable reusable recirculation container 150j to operate as a pure collection vessel (to operate without facility drain 36). A reusable container overflow line 190 extends from drain outlet 188 and includes a distal drain hook 190h that hooks to facility drain 36.

Reusable recirculation container 150j may be provided with a removable, threaded lid 192 such that it is not open to the cleanroom environment during normal operation. A hole (not illustrated) may be provided in lid 192 for receiving priming line 84 and for possibly receiving complimentary quick connect fittings on the overflow line 190 and small drain line if provided (e.g., for storage when the recirculation container is not in use). An internal “T” fitting may be placed in fluid communication with container overflow line 190, wherein one leg of the “T” fitting connects to a line extending to submersible pump (not illustrated) that is powered at the end of production shift to drain residual dialysis fluid to facility drain 36 before the operator removes threaded lid 192, pours out any remaining dialysis fluid and disinfects the inside of tank 180 of reusable recirculation container 150j with a disinfectant, such as a citric acid solution.

It should be understood that various changes and modifications to the presently preferred embodiments described herein will be apparent to those skilled in the art. It is therefore intended that such changes and modifications be covered by the appended claims. For example, while system 10 has been described in connection with supplying dialysis fluid, the systems using different concentrates or additives may be configured to provide other types of medical fluids such as saline or lactated ringers. In another example, in an alternative embodiment for system 10, certain purification and sterilization features may be modified, e.g., downstream inline filtration may be lessened and laminar flow hood 40 may be removed, and wherein filled dialysis fluid containers 90 are instead placed in an autoclave located within cargo unit 20 for a prescribed amount of time during which dialysis fluid containers 90 are steam sterilized.

1. A dialysis fluid production system comprising:
a water purification unit;
at least one concentrate;
a dialysis machine configured to receive purified water from the water purification unit and to mix the purified water with the at least one concentrate to form dialysis fluid, the dialysis machine including a drain line and a dialysis fluid return line;
a product container positioned and arranged to receive the dialysis fluid from the dialysis machine; and
a recirculation container configured to receive an end of the drain line and an end of the dialysis fluid return line, the recirculation container enabling dialysis fluid to be recirculated from the drain line, through the dialysis fluid return line, back to the dialysis machine, the recirculation container further configured to be positioned adjacent to a facility drain such that overflow dialysis fluid flows from the recirculation container into the facility drain.

2. The dialysis fluid production system of claim 1, which includes the facility drain and a cleanroom, the facility drain located within the cleanroom.

3. The dialysis fluid production system of claim 2, which includes a cargo unit configured to be transported by a vehicle, the cleanroom located within the cargo unit.

4. The dialysis fluid production system of claim 1, wherein the dialysis machine is configured (i) to recirculate dialysis fluid from the drain line, through the dialysis fluid return line, back to the dialysis machine while the product container is being filled with dialysis fluid and (ii) such that overflow dialysis fluid flows from the recirculation container into the facility drain after the product container has been filled and is being removed.

5. The dialysis fluid production system of claim 1, which includes a filter located fluidically between the dialysis machine and the product container.

6. The dialysis fluid production system of claim 1, wherein the recirculation container is configured to be positioned adjacent to the facility drain such that overflow dialysis fluid flows from the recirculation container into the facility drain by being releasably held within a recirculation fixture, the recirculation fixture configured to rest securely on the facility drain.

7. The dialysis fluid production system of claim 6, wherein the recirculation fixture includes a drain mount sized to fit over and rest securely on a top of the facility drain.

8. The dialysis fluid production system of claim 6, wherein the recirculation fixture includes a container holder formed so as to hold the recirculation container at a desired angle relative to a horizontal top of the facility drain.

9. The dialysis fluid production system of claim 8, wherein the recirculation container includes a spout, and wherein the desired angle sets the spout at about 9° above horizontal.

10. The dialysis fluid production system of claim 8, wherein the container holder includes at least one securement feature shaped to contact and orient at least one of the drain line or the dialysis fluid return line.

11. The dialysis fluid production system of claim 6, wherein the recirculation fixture is reusable and the recirculation container is disposable.

12. The dialysis fluid production system of claim 1, wherein the recirculation container includes a closed chamber, which when oriented for operation includes an upper closed chamber line for fluid communication with the facility drain and plural closed chamber lines for fluid communication with the drain line and the dialysis fluid return line.

13. The dialysis fluid production system of claim 12, wherein the closed chamber is disposable, or wherein the system is configured to run a disinfection sequence in which the closed chamber is disinfected along with the drain line and the dialysis fluid return line.

14. The dialysis fluid production system of claim 1, wherein the recirculation container includes an inner wall and an outer wall defining a dialysis fluid holding space, the outer wall extending up from the dialysis fluid holding space so as to hold the drain line above dialysis fluid residing within the dialysis fluid holding space, the inner wall forming a weir over which the dialysis fluid overflows from the dialysis fluid holding space into the facility drain.

15. The dialysis fluid production system of claim 14, wherein the weir additionally forms a mounting hook for removably fixing the recirculation container to the facility drain.

16. The dialysis fluid production system of claim 14, wherein the recirculation container includes a return tube having an end configured to connect to the dialysis fluid return line.

17. The dialysis fluid production system of claim 16, wherein the return tube extends from a bottom of the recirculation container.

18. The dialysis fluid production system of claim 1, wherein the recirculation container includes a connection structure to the facility drain, wherein the connection structure is also where dialysis fluid overflows from the recirculation container into the facility drain.

19. The dialysis fluid production system of claim 18, wherein the recirculation container includes a vacuum flask, a wash bottle, or plastic piping.

20. The dialysis fluid production system of claim 1, wherein the recirculation container includes a support fixture configured to orient the recirculation container in a desired manner relative to the facility drain.

21. The dialysis fluid production system of claim 20, wherein the recirculation container includes a beverage dispenser, a squeeze and pour container, or a tip and pour container.

22. The dialysis fluid production system of claim 1, wherein the recirculation container is configured to be cleaned and reused, the recirculation container including container connectors for fluid communication with the drain line and the dialysis fluid return line, the recirculation container further including a drain outlet for fluid communication with an overflow line configured to extend to the facility drain.

23. The dialysis fluid production system of claim 22, wherein the container connector for the dialysis fluid return line is located above the container connector for the drain line, and which includes a straw extending from the container connector for the dialysis fluid return line to a bottom of the recirculation container.

24. The dialysis fluid production system of claim 22, wherein the recirculation container includes a container line extending from the container connector for the drain line, the container line configured to connect to the drain line or the dialysis fluid return line.

25. The dialysis fluid production system of claim 22, wherein the recirculation container includes a removable lid, the removable lid including at least one of (i) a hole for receiving a priming line or (ii) at least one complimentary quick connect fitting.

26. The dialysis fluid production system of claim 1, wherein fresh dialysis fluid is pumped into a blood side of a dialyzer, through pores of a hollow fiber membranes of the dialyzer, through outsides of the hollow fiber membranes and out of a dialysis fluid side of the dialyzer at the other end of the dialyzer and to the product container, a used dialysis fluid port of dialyzer being capped to force the filtration.

27. A dialysis fluid production system comprising:
a dialysis preparation unit including a dialysis machine and a water purification unit;
at least one concentrate;
an ultrafilter;
wherein the dialysis machine is configured to receive purified water from the water purification unit and to mix the purified water with the at least one concentrate to form dialysis fluid, the dialysis machine including a fresh dialysis fluid line, a drain line and a dialysis fluid return line, wherein the fresh dialysis fluid line extends to an inlet of the ultrafilter for filtering of the fresh dialysis fluid and the dialysis fluid return line comprises an end configured for connection to a dialyzer;
a product container positioned and arranged to receive the dialysis fluid through the ultrafilter from the fresh dialysis fluid line; and
a recirculation container configured to receive an end of the drain line and the end of the dialysis fluid return line, the recirculation container enabling dialysis fluid to be recirculated from the drain line, through the dialysis fluid return line, back to the dialysis machine, the recirculation container further configured to be positioned adjacent to a facility drain such that overflow dialysis fluid flows from the recirculation container into the facility drain.

28. The dialysis fluid production system of claim 27, wherein a priming line extends from a deaeration port of the ultrafilter to facility drain, and a product line carries filtered dialysis fluid from an outlet port of the ultrafilter to the product container (90).

29. A dialysis fluid production system comprising:
a dialysis preparation unit including a dialysis machine and a water purification unit;
at least one concentrate;
an ultrafilter;
wherein the dialysis machine is configured to receive purified water from the water purification unit and to mix the purified water with the at least one concentrate to form dialysis fluid, the dialysis machine including:
a dialyzer having a blood side and a dialysis fluid side,
a fresh dialysis fluid line receiving dialysis fluid and having one removable end configured for connection to the dialysis fluid side of the dialyzer,
a dialysis fluid return line having one removable end configured for connection to the dialysis fluid side of the dialyzer,
a drain line in fluid communication with the dialysis fluid return line, and
a product container positioned and arranged to receive the dialysis fluid through the fresh dialysis fluid line; and
a recirculation container configured to receive an end of the drain line and the removable end of the dialysis fluid return line, the recirculation container enabling fluid to be recirculated from the drain line, through the dialysis fluid return line, back to the dialysis machine, the recirculation container further configured to be positioned adjacent to a facility drain such that overflow dialysis fluid flows from the recirculation container into the facility drain.

30. The dialysis fluid production system of claim 29, wherein the dialysis machine includes:
at least one mixing pump for mixing purified water from the water purification unit with the at least one concentrate,
a fresh dialysis fluid pump for delivering fresh dialysis fluid to the dialyzer; and
a used dialysis fluid pump for removing used dialysis fluid from dialyzer after treating a patient.

31. The dialysis fluid production system of claim 29, wherein the dialysis machine includes a B-concentrate pump for metering purified water through bicarbonate cartridge and A-concentrate pump for metering liquid acid concentrate from concentrate container into the mixture of purified water and bicarbonate concentrate.

32. The dialysis fluid production system of claim 29, wherein the dialysis machine includes a purified water pump that pulls purified water from the water purification unit.
